(19) United States
(12) Patent Application Publication
(10) Pub. No.: US 2010/0029933 A1
(43) Pub. Date: Feb. 4, 2010

(54) PURE FORM OF RAPAMYCIN AND A PROCESS FOR RECOVERY AND PURIFICATION THEREOF

(75) Inventors: Nitin Sopanrao Patil, Bangalore (IN); Syed Idris Hussaini, Bangalore (IN); Ashish Kumar Singh, Bihar (IN); Rakesh Bhaiyyaram Mendhe, Bhandara (IN)

Correspondence Address: FENWICK & WEST LLP, SILICON VALLEY CENTER, 801 CALIFORNIA STREET, MOUNTAIN VIEW, CA, 94041, US

(73) Assignee: Biocon Limited, Bangalore, Karnataka (IN)

(21) Appl. No.: 12/514,356

(22) PCT Filed: Dec. 26, 2006
(86) PCT No.: PCT/IN06/00502

§ 371 Date: May 11, 2009

(30) Foreign Application Priority Data
Nov. 10, 2006 (IN) 2079/CHE/2006

(51) Int. Cl. C07D 498/16; C12P 17/18

(52) U.S. Cl. 540/468; 435/119

(57) ABSTRACT

The present invention relates to a pure form of rapamycin with a total impurity content less than 1.2%; a process for recovery and purification of rapamycin comprising steps of (a) treating the fermentation broth, extracts or solutions containing rapamycin with water immiscible solvent and concentration; (b) addition of a water miscible solvent to effect separation of impurities present; (c) optionally, binding of the solvent containing the product from step (b) to an inert solid, washing the solid with a base and acid, followed by elution; (d) subjecting the elute from step (c) or the solvent containing the product from step (b) to silica gel chromatography; (e) crystallization of the product obtained from step (d); (f) subjecting a solution of the product from step (e) to hydrophobic interaction or reversed phase chromatography; and (g) re-crystallization to afford rapamycin in substantially pure form.

### FIELD OF THE INVENTION

The present invention discloses a substantially pure form of rapamycin. The invention also relates to a process for recovery and purification of rapamycin from fermentation broth, extracts or solutions containing rapamycin in a combination of steps.

### BACKGROUND AND PRIOR ART

In 1975, Vezina et al. identified (3S,6R,7E,9R,10R,12R,14S,15E,17E,19E,21S,23S,26R,27R,34aS)-9,10,12,13,14,21,22,23,24,25,26,27,32,33,34,34a-Hexadecahydro-9,27-dihydroxy-3-[(1R)-2-[(1S,3R,4R)-4-hydroxy-3-methoxycyclo hexyl]-1-methylethyl]-10,21-dimethoxy -6,8,12,14,20,26-hexamethyl-23,27-epoxy-3H-pyrido[2,1-c][1,4]oxaazacyclohentriacontine-1,5,11, 28,29(4H,6H,31H)-pentone, also known as rapamycin as well as sirolimus as an antifungal antibiotic harvested from a Streptomyces hygroscopicus culture. This culture was isolated from an Easter Island soil sample. (J. Antibiot. 28, 721-726 (1975); and U.S. Pat. No. 3,929,992, was issued to Sehgal, et. al. Dec. 30, 1975. Martel, R. et al. (1977) described the ability of this compound to inhibit the immune response (Can. J. Physiol. Pharmacol., 55, 48-51). More recently, Calne, R. Y. et al. (1989), has described rapamycin to be immunosuppressive in rats given heterotopic heart allografts (Lancet vol. 2, p. 227). Many other derivatives of these compounds as well as structural analogues have immunosuppressant property.

U.S. Pat. No. 5,508,398 discloses a process for separating a neutral non-polypeptide macrolide from acidic, basic and non-polar neutral impurities present in a concentrate of fermentation broth extracts or mother liquors containing said neutral macrolide which comprises in any order extraction step (a) and optionally one or both of steps (b) and (c), wherein (a) involves extraction with aqueous base, (b) involves extraction with aqueous acid and (c) involves treatment with non-aromatic hydrocarbon solvent.

U.S. Pat. No. 5,616,595 discloses a process for recovering water insoluble compounds (including FK506, FK520 and rapamycin) from a fermentation broth includes sequential steps of concentrating, solubilizing and diafiltering the compound of interest, all through a single closed recirculation system to recover the compound for further downstream purification.

The prior art methods for the recovery of macrolide compounds are tedious or require special setup for purification and do not result in pure product.

The inventor (Wyeth) tablets are marketed under the name of Rapamune. Rapamune tablets were analyzed by HPLC according to the method described herein and found to contain several impurities. Rapamycin is known to exist in three isomeric forms; isomer A, isomer B and isomer C. Excluding these isomers, Rapamune contained 1.2% of total impurities, 0.39% of impurity at RRT 1.34, 0.15% of impurity at RRT 0.92 and 0.24% of impurity at RRT 0.69.

The instant invention provides rapamycin in more pure form and a method to obtain the same. The present invention discloses rapamycin with total impurity content less than 1.2% obtained by HPLC. The present invention also relates to rapamycin with impurity content less than 0.15% at RRT 1.34. The present invention relates to rapamycin with impurity content less than 0.15% at RRT 0.92. The present invention relates to rapamycin with impurity content less than 0.15% at RRT 0.69.

The instant invention also relates to a process for recovery and purification of rapamycin.

### OBJECTS OF THE INVENTION

The main object of the present invention is to obtain a pure form of rapamycin with a total impurity content less than 1.2%.

Yet another object of the present invention is to obtain a pure form of rapamycin having impurity less than 0.15% at RRT 1.34, 0.92 and 0.69 min.

Yet another object of the present invention is to obtain a pure form of rapamycin through High Performance Liquid Chromatography.

Still another object of the present invention is to develop a process for recovery and purification of rapamycin from the fermentation broth.

### STATEMENT OF THE INVENTION

The present invention relates to a pure form of rapamycin with a total impurity content less than 1.2%; a process for recovery and purification of rapamycin comprising steps of (a) treating the fermentation broth, extracts or solutions containing rapamycin with water immiscible solvent and concentration; (b) addition of a water miscible solvent to effect separation of impurities present; (c) optionally, binding of the solvent containing the product from step (b) to an inert solid, washing the solid with a base and acid, followed by elution; (d) subjecting the elute from step (c) or the solvent containing the product from step (b) to silica gel chromatography; (e) crystallization of the product obtained from step (d); (f) subjecting a solution of the product from step (e) to hydrophobic interaction or reversed phase chromatography; and (g) re-crystallization to afford rapamycin in substantially pure form.

### BRIEF DESCRIPTION OF ACCOMPANYING DRAWINGS

FIG. 1 HPLC chromatogram of Rapamune

FIG. 2 HPLC chromatogram of purified Rapamycin

### DETAILED DESCRIPTION OF THE INVENTION

The present invention relates to a pure form of rapamycin with a total impurity content less than 1.2%.

In yet another embodiment of the present invention, the rapamycin having impurity less than 0.15% at RRT 1.34, 0.92 and 0.69 min.

In still another embodiment of the present invention, the rapamycin is having a purity ranging between 98.8% to 100%.

In still another embodiment of the present invention, the rapamycin is having a purity preferably 98.8%.

In still another embodiment of the present invention, the rapamycin is produced by fermentation broth.

In still another embodiment of the present invention, the rapamycin is obtained by High Performance Liquid Chromatography.

In still another embodiment of the present invention, the rapamycin is in crystalline form.

The present invention also relates to a process for recovery and purification of rapamycin comprising steps of:
a) treating the fermentation broth, extracts or solutions containing rapamycin with water immiscible solvent and concentration;
b) addition of a water miscible solvent to effect separation of impurities present;
c) optionally, binding of the solvent containing the product from step (b) to an inert solid, washing the solid with a base and acid, followed by elution;
d) subjecting the elute from step (c) or the solvent containing the product from step (b) to silica gel chromatography;
e) crystallization of the product obtained from step (d);
f) subjecting a solution of the product from step (e) to hydrophobic interaction or reversed phase chromatography; and
g) re-crystallization to afford rapamycin in substantially pure form.

In still another embodiment of the present invention, water immiscible solvent is selected from a group comprising hydrocarbons, heterocyclic compounds, ethers and esters.

In still another embodiment of the present invention, water immiscible solvent is selected from a group comprising benzene, toluene, butanol, dichloromethane, chloroform, ethyl acetate, isobutyl acetate and butyl acetate.

In still another embodiment of the present invention, water immiscible solvent is ethyl acetate.

In still another embodiment of the present invention, water miscible solvent is selected from a group comprising water, alcohols, ketones and dielectric aprotic solvents.

In still another embodiment of the present invention, water miscible solvent is selected from a group comprising water, methanol, ethanol, isopropyl alcohol, acetone and acetonitrile.

In still another embodiment of the present invention, inert solid is selected from a group comprising diatomaceous earth, sand, activated charcoal, silica gel and polymeric resin.

In still another embodiment of the present invention, inert solid is diatomaceous earth.

In still another embodiment of the present invention, inert solid is activated charcoal.

In still another embodiment of the present invention, the base used is either an organic or inorganic base.

In still another embodiment of the present invention, the base used is an inorganic base.

In still another embodiment of the present invention, the base is sodium bicarbonate.

In still another embodiment of the present invention, the acid used is either an organic or inorganic acid.

In still another embodiment of the present invention, the acid used is an inorganic acid.

In still another embodiment of the present invention, the acid is hydrochloric acid.

In still another embodiment of the present invention, elution is carried out using an organic solvent selected from a group comprising acetone, ethyl acetate, chloroform, dichloromethane, hexane, petroleum ether, methanol and diethyl ether or mixtures thereof.

In still another embodiment of the present invention, elution is carried out using acetone.

In still another embodiment of the present invention, crystallization is carried out using ethers.

In still another embodiment of the present invention, crystallization is carried out using diethyl ether.

In still another embodiment of the present invention, hydrophobic interaction chromatography is carried out with a polymeric resin selected from a group comprising polystyrene, poly(styrene-divinyl benzene), poly(acrylate) and poly(methacrylate).

In still another embodiment of the present invention, reversed phase chromatography is carried out with a resin selected from a group comprising C4, C8 and C18 bonded silica.

In still another embodiment of the present invention, elution in hydrophobic interaction or reversed phase chromatography is carried out using solvents selected from a group comprising methanol, acetone, acetonitrile, water, ethanol, propanol, butanol and tetrahydrofuran or mixture thereof.

In still another embodiment of the present invention, re-crystallization is carried out using organic solvent selected from a group comprising acetonitrile, acetone, methanol, ethanol, propanol, butanol, chloroform, dichloromethane, ethyl acetate, hexane and heptane.

In still another embodiment of the present invention, the purified product is either one of the isomeric forms of rapamycin namely isomer A, isomer B or isomer C.

In still another embodiment of the present invention, the purified product is isomer B of rapamycin.

The present invention relates to rapamycin with total impurity content less than 1.2% by HPLC. The present invention also relates to rapamycin with impurity content less than 0.15% at RRT 1.34. LC-MS analysis of rapamune as well as rapamycin from present invention shows that the impurity at RRT 1.34 gives peak at m/z of 951 corresponding to [M+Na]⁺. This impurity has mass 14 Da more than that of rapamycin. The impurity at RRT 0.69 or RRT 0.92 present in rapamycin produced using the instant process is less than 0.15% each. All RRTs here are with respect to isomer B of rapamycin.

The HPLC method used herein for analysis of Rapamune and rapamycin purified according to the present invention is as:

Column: Agilent Eclipse XDB-C8, 3.5 μm, diameter-4.6 mm, length-150 mm
Flow rate: 1.5 ml/min
Detection wavelength: 287 mm
Injection volume: 20 μl

### Diluent: Acetonitrile

### Temperature: 45° C.

Approximate retention time of isomer B of rapamycin: 26 min

Mobile phase: Buffer A-acetonitrile; Buffer B-2 mM KH₂PO₄ in water. The gradient is as given in Table 1.

TABLE 1

| Time (min) | Buffer A (%) | Buffer B (%) |
|---|---|---|
| 0 | 43 | 57 |
| 3 | 43 | 57 |
| 5 | 51 | 49 |
| 50 | 52 | 48 |
| 55 | 80 | 20 |
| 58 | 43 | 57 |
| 60 | 43 | 57 |

The instant invention also relates to a process for recovery and purification of rapamycin comprising:
a) treating the fermentation broth, extracts or solutions containing rapamycin with water immiscible solvent and concentration,
b) addition of a water miscible solvent to effect separation of impurities present,
c) optionally, binding of the solvent containing the product from step (b) to an inert solid, washing the solid with a base and acid, followed by elution
d) subjecting the elute from step (c) or the solvent containing the product from step (b) to silica gel chromatography
e) crystallization of the product obtained from step (d).
f) subjecting a solution of the product from step (e) to hydrophobic interaction or reversed phase chromatography
g) re-crystallization to afford rapamycin in substantially pure form.

Rapamycin of the present invention is produced by fermentation. The broth obtained by fermentation can be directly extracted by water immiscible solvent. The water immiscible solvent may be selected from ethyl acetate, toluene, butyl acetate, isobutyl acetate, butanol, benzene, chloroform and dichloromethane. Any crude material in solid, semisolid or liquid form obtained from broth can be treated with water immiscible solvent to effect solubilization of rapamycin into the water immiscible solvent. The water immiscible solvent containing rapamycin can be concentrated. The concentration can be affected by methods known. The concentration can be affected by vaporization of the solvent. The vaporization of the solvent can be carried out by heating without or with reduced pressure. The concentrate can be treated with a solvent to effect separation of impurities present with rapamycin. The impurities may be present in form of solid or liquid, immiscible with the solvent or both. The impurities can be separated out by filtration, phase separation or both. The solvent can be a water miscible solvent. Preferably, the solvent can be selected from acetone, methanol, or acetonitrile. Optionally, the concentrate is bound to an inert solid and washed with a base and/or acid. Rapamycin is then eluted with an organic solvent. The base and acid can be selected from an inorganic or organic bases and acids; Preferably, the base can be aqueous sodium bicarbonate and the acid can be aqueous hydrochloric acid. The organic solvent can be chosen from the solvents that are able to dissolve rapamycin and mixtures thereof. The elute then can be concentrated.

The concentrate can be subjected to silica gel chromatography. The elution may be carried out with one of the solvents from acetone, ethyl acetate, chloroform, dichloromethane, hexane, heptane, petroleum ether, methanol, and diethyl ether or mixture thereof. The product containing fractions from the chromatography can be mixed and concentrated. The concentrate can be treated with a solvent to isolate the product. The product can be filtered and dried. Optionally, this solvent treatment may be repeated.

The product can be subjected to a hydrophobic interaction chromatography or reversed phase chromatography. The hydrophobic interaction chromatography may be carried out with a polymeric resin. This polymeric resin may be selected from polystyrene, poly(styrene-divinyl benzene), poly(acrylate) and poly(methacrylate). The resin for reversed phase chromatography may be selected from C4, C8 or C18 bonded silica. The eluting solvent for hydrophobic interaction chromatography or reversed phase chromatography can be selected from methanol, acetone, acetonitrile, water, ethanol, propanol, butanol and tetrahydrofuran or mixture thereof. The fractions containing product with desired purity can be mixed, concentrated, extracted with a water immiscible solvent. The extract can be concentrated.

The concentrate or the product obtained after the hydrophobic interaction chromatography or reversed phase chromatography can be re-crystallized from an organic solvent. This solvent may be selected from acetone, acetonitrile, methanol, ethanol, propanol, ethyl acetate, chloroform and dichloromethane.

The invention is further elaborated with the help of following examples. However, these examples should not be construed to limit the scope of the invention.

### Example 1

### Recovery of Rapamycin

The fermentation broth (11 Kg) containing rapamycin was twice extracted with 11 L of ethyl acetate. The ethyl acetate extract was concentrated to obtain 206 g of oily residue. The residue was extracted thrice with 600 ml of acetonitrile. The acetonitrile extracts were concentrated to obtain 90 g of oily residue. The residue was mixed with 1 L of ethyl acetate. 500 g of diatomaceous earth was added to this solution. The solution was concentrated completely. The concentrate was slurried in 1 L of 0.01 M sodium bicarbonate solution in water. The mixture was filtered. The filtered solids were further washed with 9 L of 0.01 M sodium bicarbonate solution. The base wash was followed by 10 L of 0.1 N aqueous hydrochloric acid solution. The solids were then washed with water. The product was eluted using ethyl acetate. The elute was concentrated to obtain 56 g of residue.

The residue was applied to a column packed with silica gel. The column was washed with 15% acetone in hexane and 25% acetone in hexane. The product was eluted with 40% acetone in hexane. The product containing fractions were concentrated to obtain 23 g of residue. The residue was mixed with diethyl ether and the mixture was stirred at 4° C. The mixture was filtered to isolate crystals of rapamycin. The crystals were dried to obtain 6 g of white powder with ˜95% purity.

### Example 2

### Recovery of Rapamycin

The fermentation broth (2500 Kg) containing rapamycin was extracted with ethyl acetate (three extractions in the ratio of 1:0.5, 1:0.25, 1:0.25). The ethyl acetate extract was concentrated to about 1000 Kg. The partially concentrated ethyl acetate layer was washed with water. The ethyl acetate layer was concentrated to obtain 50 Kg of oily residue. The residue was extracted thrice with 150 Kg of acetonitrile. The acetonitrile extracts were concentrated to obtain 11 Kg of oily residue. The residue was mixed with 200 Kg of ethyl acetate. 0.765 Kg of activated charcoal was added to this solution. The solution was stirred and filtered. The filtrate was concentrated completely to obtain residue.

The residue was applied to a column packed with silica gel. The column was washed with 15% acetone in hexane and 25% acetone in hexane. The product was eluted with 40% acetone in hexane. The product containing fractions were concentrated to obtain oily residue. The residue was mixed with 200 Kg of ethyl acetate. 0.765 Kg of activated charcoal was added to this solution. The solution was stirred, filtered and concentrated. The concentrate was mixed with diethyl ether and the mixture was stirred at 4° C. The mixture was filtered to isolate crystals of rapamycin. The crystals were dried to obtain 1.1 Kg of white powder with ˜90% purity.

### Example 3

### Purification of Rapamycin

3 g of powder obtained in Example 1 was dissolved in 90 ml of acetonitrile. The solution was concentrated and kept at 4° C. for crystallization. The crystals were filtered and dried. 2.5 g of white crystals were obtained. The total impurities in these crystals were 0.5% and the impurity at RRT 1.34 was 0.25%.

### Example 4

### Purification of Rapamycin

7 g of powder obtained in Example 2 was dissolved in acetonitrile at a concentration of 150 mg/ml. The solution was loaded on a column packed with C8-bonded silica. The column diameter was 100 mm and length was 250 mm. The product was eluted with a mobile phase of acetonitrile and water in the ratio of 60:40. The fractions containing pure product were pooled and concentrated. The concentrate was extracted with ethyl acetate. The ethyl acetate layer was concentrated. To the concentrate, 200 ml of acetonitrile was added. The solution was concentrated and kept at 4° C. for crystallization. The crystals were filtered and dried. 1.8 g of white powder was obtained. The total impurities in this powder were 0.15%. The impurities at RRTs 1.34 and 0.92 were 0.07% and 0.03% respectively. The impurity at RRT 0.69 was not detected.

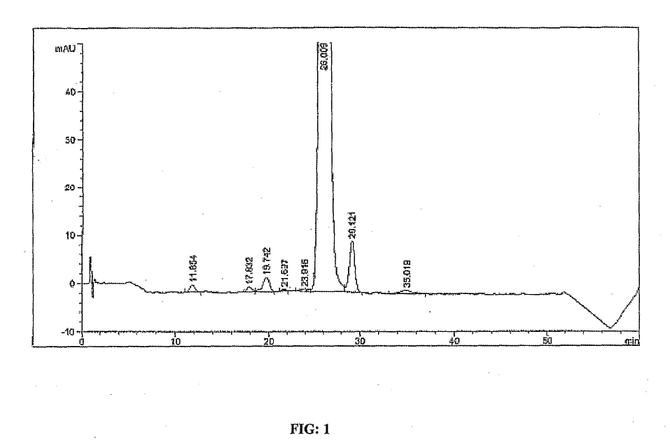

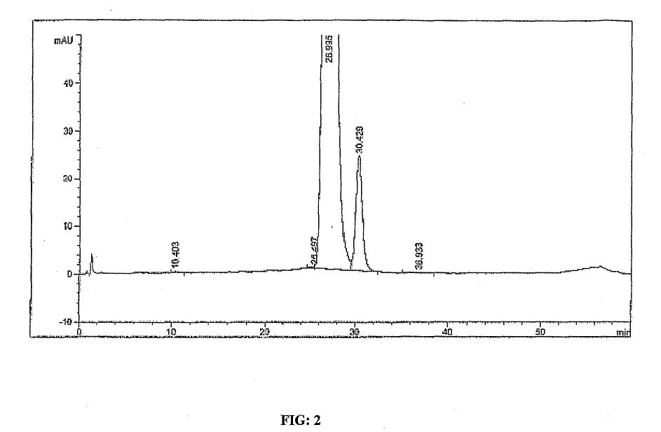

The HPLC chromatograms for Rapamune and that of rapamycin obtained as above is shown in FIG. 1 and FIG. 2 respectively. The details of chromatogram as in FIG. 1 is given in Table 2 and that of FIG. 2 is given in Table 3. Comparison of FIG. 1 and FIG. 2 and the corresponding tables shows that the rapamycin obtained from the instant invention is substantially pure.

TABLE 2

| Peak | Ret Time | | Width | Area | | |
|---|---|---|---|---|---|---|
| No. | [min] | Type | [Min] | [mAU * s] | Area % | Name |
| 1 | 11.854 | MM | 0.6582 | 58.69173 | 0.3543 | |
| 2 | 17.832 | MM | 0.6259 | 39.30749 | 0.2373 | |
| 3 | 19.742 | FM | 0.8392 | 149.03468 | 0.8997 | Isomer A |
| 4 | 21.697 | MM | 0.5363 | 11.47231 | 0.0693 | |
| 5 | 23.916 | MF | 1.0037 | 25.46312 | 0.1537 | |
| 6 | 26.009 | MF | 1.0007 | 1.57807e4 | 95.2694 | Isomer B |
| 7 | 29.121 | FM | 0.6897 | 434.99039 | 2.6261 | Isomer C |
| 8 | 35.019 | MM | 1.4984 | 64.62498 | 0.3901 | |
| Totals: | | | | 1.65643e4 | | |

TABLE 3

| | Ret Time | | | Area | Height | |
|---|---|---|---|---|---|---|
| Peak No. | [min] | Type | Width [Min] | [mAU * s] | [mAU] | Area % |
| 1 | 10.403 | MM | 0.5934 | 9.29993 | 2.61204e−1 | 0.0383 |
| 2 | 25.497 | MF | 0.3648 | 7.24601 | 3.31032e−1 | 0.0298 |
| 3 | 26.995 | MF | 1.2051 | 2.30411e4 | 318.66272 | 94.8328 |
| 4 | 30.429 | FM | 0.8420 | 1221.24658 | 24.17310 | 5.0264 |
| 5 | 36.933 | MM | 1.7911 | 17.66060 | 1.64340e−1 | 0.0727 |
| Totals: | | | | 2.42965e4 | 343.59240 | |

### Example 5

### Purification of Rapamycin

7 g of powder obtained in Example 2 was dissolved in 175 ml of acetone. To this, 175 ml of water was added. The solution was passed through a column packed with HP20SS resin. The column diameter was 20 mm and length was 1 m. The column was washed with 50% acetone in water and 60% acetone in water. The elution was carried out with 70% acetone in water. The fractions containing pure product were pooled and concentrated. The concentrate was extracted with ethyl acetate. The ethyl acetate layer was concentrated. To the concentrate, 200 ml of acetonitrile was added. The solution was concentrated and kept at 4° C. for crystallization. The crystals were filtered and dried. 1.6 g of white powder was obtained. The total impurities in this powder were 0.45% and the impurities at RRTs 1.34, 0.92 and 0.68 were 0.03%, 0.14% and 0.13%, respectively.

1. A pure form of rapamycin with a total impurity content less than 1.2% wherein said rapamycin having impurity less than 0.15% at RRT 1.34, 0.92 and 0.69 min.

2. (canceled)

3. (canceled)

4. The rapamycin as claimed in claim 1, wherein said rapamycin is having a purity preferably 98.8%.

5. The rapamycin as claimed in claim 1, wherein said rapamycin is produced by fermentation broth.

6. The rapamycin as claimed in claim 1, wherein said rapamycin is obtained by High Performance Liquid Chromatography.

7. The rapamycin as claimed in claim 1, wherein said rapamycin is in crystalline form.

8. A Process for recovery and purification of rapamycin comprising:
a) treating the fermentation broth, extracts or solutions containing rapamycin with water immiscible solvent and concentration;
b) addition of a water miscible solvent to effect separation of impurities present;
c) optionally, binding of the solvent containing the product from step (b) to an inert solid, washing the solid with a base and acid, followed by elution;
d) subjecting the elute from step (c) or the solvent containing the product from step (b) to silica gel chromatography;
e) crystallization of the product obtained from step (d);
f) subjecting a solution of the product from step (e) to hydrophobic interaction or reversed phase chromatography; and
g) re-crystallization to afford rapamycin in substantially pure form.

9. The process as claimed in claim 8, wherein the water immiscible solvent is selected from a group comprising hydrocarbons, heterocyclic compounds, ethers and esters.

10. The process as claimed in claim 8, wherein the water immiscible solvent is selected from a group comprising benzene, toluene, butanol, dichloromethane, chloroform, ethyl acetate, isobutyl acetate and butyl acetate.

11. The process as claimed in claim 8, wherein the water immiscible solvent is ethyl acetate.

12. The process as claimed in claim 8, wherein the water miscible solvent is selected from a group comprising water, alcohols, ketones and dielectric aprotic solvents.

13. The process as claimed in claim 8, wherein the water miscible solvent is selected from a group comprising water, methanol, ethanol, isopropyl alcohol, acetone and acetonitrile.

14. The process as claimed in claim 8, wherein the inert solid is selected from a group comprising diatomaceous earth, sand, activated charcoal, silica gel and polymeric resin.

15. The process as claimed in claim 8, wherein the inert solid is diatomaceous earth.

16. The process as claimed in claim 8, wherein the inert solid is activated charcoal.

17. The process as claimed in claim 8, wherein the base used is either an organic or inorganic base.

18. The process as claimed in claim 8, wherein the base used is an inorganic base.

19. The process as claimed in claim 8, wherein the base is sodium bicarbonate.

20. The process as claimed in claim 8, wherein the acid used is either an organic or inorganic acid.

21. The process as claimed in claim 8, wherein the acid used is an inorganic acid.

22. The process as claimed in claim 8, wherein the acid is hydrochloric acid.

23. The process as claimed in claim 8, wherein the elution is carried out using an organic solvent selected from a group comprising acetone, ethyl acetate, chloroform, dichloromethane, hexane, petroleum ether, methanol and diethyl ether or mixtures thereof.

24. The process as claimed in claim 8, wherein the elution is carried out using acetone.

25. The process as claimed in claim 8, wherein the crystallization is carried out using ethers.

26. The process as claimed in claim 8, wherein the crystallization is carried out using diethyl ether.

27. The process as claimed in claim 8, wherein the hydrophobic interaction chromatography is carried out with a polymeric resin selected from a group comprising polystyrene, poly(styrene-divinyl benzene), poly(acrylate) and poly(methacrylate).

28. The process as claimed in claim 8, wherein the reversed phase chromatography is carried out with a resin selected from a group comprising C4, C8 and C18 bonded silica.

29. The process as claimed in claim 8, wherein the elution in hydrophobic interaction or reversed phase chromatography is carried out using solvents selected from a group comprising methanol, acetone, acetonitrile, water, ethanol, propanol, butanol and tetrahydrofuran or mixture thereof.

30. The process as in claim 8, wherein the re-crystallization is carried out using organic solvents selected from a group comprising acetonitrile, acetone, methanol, ethanol, propanol, butanol, chloroform, dichloromethane, ethyl acetate, hexane and heptane.

31. The process as claimed in claim 8, wherein the purified product is either one of the isomeric forms of rapamycin namely isomer A, isomer B or isomer C.

32. The process as claimed in claim 8, wherein the purified product is isomer B of rapamycin.
